(19) United States
(12) Patent Application Publication
(10) Pub. No.: US 2013/0281361 A1
(43) Pub. Date: Oct. 24, 2013

(54) METHODS OF TREATING AND PREVENTING RHINOVIRUS INFECTION

(71) Applicants: David L. Hava, Natick, MA (US); Robert W. Clarke, Medfield, MA (US)

(72) Inventors: David L. Hava, Natick, MA (US); Robert W. Clarke, Medfield, MA (US)

(73) Assignee: PULMATRIX, INC., Lexington, MA (US)

(21) Appl. No.: 13/883,912

(22) PCT Filed: Nov. 4, 2011
(86) PCT No.: PCT/US2011/059330

§ 371 Date: Jun. 24, 2013

Related U.S. Application Data
(60) Provisional application No. 61/411,092, filed on Nov. 8, 2010.

(51) Int. Cl. A61K 38/17

(52) U.S. Cl. CPC A61K 38/1729
USPC 514/3.7

(57) ABSTRACT

The present invention relates to methods of treating or preventing non-enveloped viral infection and/or disease caused by non-enveloped virus infection, particularly a viral infection caused by rhino virus. The methods comprise administering to an individual in need thereof an effective amount of a defensin peptide. The defensin peptide may be selected from α-, β-, or θ-defensins.

### RELATED APPLICATIONS

This application claims the benefit of U.S. Provisional Application No. 61/411,092, filed on Nov. 8, 2010, the entire teaching of which is incorporated herein by reference.

### BACKGROUND

The innate immune system is the first line of defense against invading pathogens. Innate immunity includes physical (e.g., barriers), cellular and molecular components that act broadly to protect against bacterial, viral and fungal pathogens. These defenses include antimicrobial peptides and proteins secreted by leukocytes and epithelial cells that may be active against bacterial and viral pathogens. Human antimicrobial proteins include lactoferrin, lysozyme, secretory leukocyte protease inhibitor (SLPI), complement factors, LL-37, and defensins. The defensins are further classified into α-defensins, which are secreted by neutrophils, paneth cells, and epithelial cells (intestinal, airway, and female genitourinary tract) and β-defensins which are primarily secreted by epithelial cells (Ganz, T., Nat Rev Immunol 3(9):710-720, 2003). There are six human α-defensins, termed human neutrophil proteins (HNPs): HNP1-4 and human defensins 5 and 6 (HD5 and HD6). Although numerous human 13-defensin (HBD) genes have been found in the human genome, only four peptides (HBD1-4), have been characterized. Interestingly, a third class of defensins, the θ-defensins, are present in the human genome, but they are unexpressed pseudogenes. Synthetic θ-defensins (termed retrocyclins) based on the pseudogenes have been made in vitro and shown to have antiviral activity (Cole et al., Proc Natl Acad Sci 99(4):1813-8, 2002).

A principle mechanism of action for antimicrobial peptides is the direct disruption of microbial membranes (Brogden, K., Nat Rev Microbiol 3(3):238-50, 2005). Recent evidence indicates that multiple mechanisms are involved in the antiviral effect of defensins and these mechanisms depend on the defensin, virus and target cell (Ding et al. J Innate Immun 1:413-420, 2009). For example, θ defensin has been shown to inhibit viral replication of HIV, an enveloped virus, in HeLa cells (Maury, W., et al., U.S. Patent Appl'n Publication No. 20030022829). It has been further demonstrated that a subset of human antimicrobial peptides has activity against non-enveloped viruses like adenovirus, herpes virus (HSV) and papillomavirus, and this antiviral activity is postulated to act through inhibition of viral entry (Buck, C. et al., Proc Natl Acad Sci 103(5):1516-21, 2006; Hazrati, E. et al., J Immunol 177(12):8658-66, 2006; Smith, J. and Nemerow, G., Cell Host Microbe 3(1):11-19, 2008). For adenovirus and papillomavirus, antiviral activity was observed for HNP1, HD5 and HBD-1, but not for HBD-2. In addition, the α-defensins (HNP1-4, HD-5, and HD-6) and HBD-3 exhibited antiviral activity against HSV.

Porcine β-defensins have been postulated to be effective to treat or prevent viral diseases caused by various viral families, including the Picornaviridae family (Elahi, S., et al., U.S. Patent Appl'n Publication No. 20060008466). However, human β-defensins have not been shown to have antiviral activity against single-stranded RNA non-enveloped viruses, such as rhinovirus of the Picornaviridae family. In fact, HBD-2 was observed to be unable to inactivate rhinovirus (Proud et al., J Immunol 172:4637-4645, 2004).

A large number of people are affected each year with non-enveloped viral infections, including rhinovirus infections. Rhinovirus is most commonly associated with the common cold and nasal infections, but rhinovirus is also known to play a significant role in acute otitis media and asthma exacerbations (Pitkäranta, A., et al., Pediatrics 102(2 Pt 1):291-5, 1998; Friedlander, S. L. & Busse, W. W., J Allergy Clin Immunol 116(2):267-73, 2005). Although many antimicrobial medicines are available to treat bacterial infections, very few antiviral medicines have been developed. Therefore, new therapies for the treatment, prevention, management, and/or amelioration of non-enveloped viral infections and symptoms thereof, especially rhinovirus, are needed.

### SUMMARY

The invention relates to methods for treating or preventing non-enveloped viral infections, such as rhinovirus infection and/or disease caused by non-enveloped viral infection, comprising administering an effective amount of a defensin peptide to an individual in need thereof. In one aspect, the non-enveloped virus is a rhinovirus. In a preferred aspect, the rhinovirus is human rhinovirus.

In yet a further particular aspect, the invention also relates to methods for the treatment or prevention of acute exacerbations of chronic respiratory diseases, such as asthma, airway hyperresponsiveness, seasonal allergic allergy, bronchiectasis, chronic bronchitis, emphysema, chronic obstructive pulmonary disease, cystic fibrosis and the like, comprising administering an effective amount of a defensin peptide to an individual in need thereof.

The defensin peptide can be a precursor or pro-defensin peptide or can be a mature defensin peptide.

In some embodiments, the defensin peptide is a precursor α-defensin peptide, β-defensin peptide, or combinations thereof. In other embodiments, the defensin peptide is a mature α-defensin peptide, β-defensin peptide, θ-defensin peptide, or combinations thereof. In further embodiments, the defensin peptide is a human α-defensin peptide, β-defensin peptide, θ-defensin peptide, or combinations thereof.

In particular embodiments, the defensin peptide is precursor human neutrophil peptide 1 (HNP1), precursor human neutrophil peptide 2 (HNP2), precursor human neutrophil peptide 3 (HNP3), precursor human neutrophil peptide 4 (HNP4), precursor human defensin 5 (HD5), precursor human defensin 6 (HD6), precursor human β-defensin 1 (HBD-1), precursor human β-defensin 2 (HBD-2), precursor human β-defensin 3 (HBD-3), precursor human β-defensin 4 (HBD-4), and combinations thereof. In further particular embodiments, the defensin peptide is mature human neutrophil peptide 1 (HNP1), mature human neutrophil peptide 2 (HNP2), mature human neutrophil peptide 3 (HNP3), mature human neutrophil peptide 4 (HNP4), mature human defensin 5 (HD5), mature human defensin 6 (HD6), mature human β-defensin 1 (HBD-1), mature human β-defensin 2 (HBD-2), mature human β-defensin 3 (HBD-3), mature human β-defensin 4 (HBD-4), and combinations thereof. In yet further particular embodiments, the defensin peptide is human θ-defensin 1 (retrocyclin-1), human θ-defensin 2 (retrocyclin-2), or combinations thereof.

The defensin peptide(s) can administered as a component of a pharmaceutical composition. In some embodiments, the pharmaceutical composition of the invention consists essentially of a defensin peptide. The pharmaceutical composition can be administered by inhalation or intranasally.

### BRIEF DESCRIPTION OF THE DRAWINGS

FIG. 1 is a graph illustrating that human β-defensins, HBD-1, HBD-2, HBD-3 and HBD-4 exhibited antiviral activity against rhinovirus (Rv16).

FIG. 2 is a graph illustrating that human α-defensins, HNP-1 (α-1), HNP-3 (α-3) and HNP-5 (α-5) exhibited antiviral activity against rhinovirus (Rv16) while cathelicidin (LL-37/hCAP-18) had little effect.

### DETAILED DESCRIPTION

As described and exemplified herein, the inventors have discovered that human defensin peptides possess antiviral activity against non-enveloped viruses. This discovery was surprising and is contrary to a published study reporting that defensin polypeptides are not effective against rhinovirus, which is a non-enveloped virus. See, e.g., Proud et al., J Immunol 172:4637-4645, 2004. Thus, the invention provides methods for treating or preventing non-enveloped viral infections, such as rhinovirus infection, comprising administering an effective amount of a defensin peptide to an individual in need thereof. The defensin peptide can be part of a pharmaceutical formulation.

To facilitate the preparation of a clear and concise specification, the invention is further described with reference to preferred embodiments of treating non-enveloped viral infections. It is intended and to be understood that the methods can be used to treat or prevent a non-enveloped viral infection in any tissue.

### Definitions

Defensin peptides referred to herein as “isolated” are peptides purified to a state beyond that in which they exist in mammalian cells. “Isolated” defensin peptides include proteins and peptides obtained from biological sources (e.g., blood) by any suitable method, including essentially pure peptides, peptides produced by chemical synthesis (e.g., synthetic peptides), or by combinations of biological and chemical methods, and recombinant peptides which are isolated. The peptides can be obtained in an isolated state of at least about 50% by weight, preferably at least about 75% by weight, and more preferably, in essentially pure form. Defensin peptides referred to herein as “recombinant peptides” are proteins and peptides produced by the expression of recombinant nucleic acids.

As used herein “defensin peptide” refers to naturally occurring or endogenous defensin peptides, to synthetic defensin peptides, to peptides having an amino acid sequence which is the same as that of a naturally occurring or endogenous corresponding defensin peptide (e.g., recombinant proteins), and to functional variants of each of the foregoing (e.g., functional fragments and/or mutants produced via mutagenesis and/or recombinant techniques). Accordingly, as defined herein, the term includes mature active protein, glycosylated or unglycosylated defensin proteins, polymorphic or allelic variants, lipidated (e.g., pegylated) defensin peptides, and other isoforms of defensins (e.g., produced by alternative splicing, proteolytic cleavage, or other cellular processes), and functional fragments. “Mature active protein” refers to a defensin peptide that has been processed to remove the pre-, pro- or prepro- sequence.

Naturally occurring or endogenous defensin peptides includes wild type proteins and peptides such as mature active defensin peptides, polymorphic or allelic variants and other isoforms which occur naturally in mammals (e.g., humans). Such proteins and peptides can be recovered from a source which naturally produces defensins. These proteins and peptides having the same amino acid sequence as a naturally occurring or endogenous corresponding defensin are referred to by the name of the corresponding mammal. For example, where the corresponding mammal is a human, the protein is designated as a human defensin peptide (e.g., a recombinant human defensin peptide produced in a suitable host cell).

“Functional variants” of defensin peptides include functional fragments or portions, functional mutant, and/or functional fusion proteins or peptides. Generally, fragments or portions of defensin peptides encompassed by the present invention include those having a deletion (i.e., one or more deletions) of an amino acid (i.e., one or more amino acids) relative to the mature active defensin (such as N-terminal, C-terminal or internal deletions). Fragments or portions in which only contiguous amino acids have been deleted or in which non-contiguous amino acids have been deleted relative to mature active defensin peptides are also envisioned. For example, amino-terminal and/or carboxy-terminal modifications are included in the present invention.

A defensin peptide or variant has an amino acid sequence which is at least about 55% similar, about 65% similar, about 75% similar, about 80% similar, at least about 90% similar, at least about 95% similar, at least about 96% similar, at least about 97% similar, at least about 98% similar, at least about 99% similar, or 100% similar to SEQ ID NO: 1, 2, 3, 4, 5, 6, 7, 8, 9, 10, 11 or 12.

Generally, mutants or derivatives of defensin peptides encompassed by the present invention include natural or non-naturally occurring (e.g., synthetic) variants differing by the addition, deletion and/or substitution of one or more contiguous or non-contiguous amino acid residues, or modified proteins or peptides in which one or more residues is modified, and mutants comprising one or more modified residues. Preferred mutants are natural or non-naturally occurring variants of defensin peptides differing by the addition, deletion and/or substitution of one or more contiguous or non-contiguous amino acid residues.

A “functional fragment or portion,” “functional mutant” and/or “functional fusion protein or peptide” of a defensin peptide refers to an isolated and/or recombinant protein or oligopeptide which has at least one property, activity and/or function characteristic of a defensin peptide, such as inhibiting viral infection.

The defensin peptides of the present invention also include the cyclic, amidated, acetylated, sulfated, phosphorylated, glycosylated, and oxidized derivatives of the amino acid sequences described herein. As used herein, the term “amino acid” is used in its broadest sense to mean the naturally occurring amino acids as well as non-naturally occurring amino acids, including amino acid analogs. Thus, reference herein to an amino acid includes, for example, naturally occurring proteogenic (L)-amino acids, as well as (D)-amino acids, chemically modified amino acids such as amino acid analogs, naturally occurring non-proteogenic amino acids such as norleucine, and chemically synthesized compounds having properties known in the art to be characteristic of an amino acid. As used herein, the term “proteogenic” indicates that the amino acid can be incorporated into a protein in a cell through a metabolic pathway.

Suitable fragments or mutants can be identified by screening. Where the resulting protein or peptide displays activity in the assay, the resulting protein or peptide (“fragment”) is functional.

### Therapeutic Methods and Use

In one aspect, the invention is a method for treating or preventing non-enveloped viral infections or disease caused by non-enveloped viral infections, comprising administering an effective amount of a defensin peptide to an individual in need thereof. The defensin peptide can be administered to the individual by any suitable route of administration. Preferably, the route of administration results in defensin peptide accumulating in tissue(s) where non-enveloped viruses enter host cells and/or replicate in an amount sufficient to inhibit viral entry and/or replication. Generally, the defensin peptide is administered as a component of a pharmaceutically acceptable composition. Suitable pharmaceutically acceptable compositions and modes of administration are described herein.

The individual to be treated in accordance with the invention may have a diagnosed non-enveloped viral infection, such as an infection diagnosed by clinical exam, for example with a chest x-ray or CT scan, and/or confirmed presence of a non-enveloped viral infection (e.g., using a suitable microbiological or molecular diagnostic test). The individual to be treated in accordance with the invention may be at risk for a non-enveloped viral infection. Generally, such individuals are exposed to non-enveloped viruses more frequently than the general population, or are more susceptible to non-enveloped viral infection than the general population. Individuals at risk for a non-enveloped viral infection include, for example, health care workers, individuals who care for infected family members, individuals with chronic lung diseases (e.g., asthma, chronic bronchitis, emphysema, chronic obstructive pulmonary disease, cystic fibrosis, bronchiectasis), individuals who are immunosuppressed, infants, newborns and young (e.g., humans younger than about 12 years of age), and elderly (e.g., humans older that about 65 or 70 years of age).

Accordingly, in some embodiments, the invention is a method for the treatment or prevention of a non-enveloped viral infection in an individual with a chronic underlying respiratory disease, such as asthma, chronic bronchitis, emphysema, chronic obstructive pulmonary disease (COPD), cystic fibrosis (CF), bronchiectasis, or rhinitis comprising administering an effective amount of a defensin peptide formulation to the individual.

In other embodiments, the invention also relates to methods for the treatment or prevention of acute exacerbations of respiratory diseases or disorders, such as asthma, seasonal allergic asthma, airway hyperresponsiveness, allergic rhinitis, seasonal or perennial rhinitis, rhinoconjunctivitis, bronchiectasis, chronic bronchitis, emphysema, chronic obstructive pulmonary disease, cystic fibrosis and the like, comprising administering an effective amount of a defensin peptide to an individual in need thereof.

The defensin peptide administered in accordance with the methods of the invention can be an α-defensin, a β-defensin, a θ-defensin, a functional variant of any of the foregoing, or combinations thereof. In some embodiments, the defensin peptide is an α-defensin, e.g., a mature α-defensin. In further embodiments, the defensin peptide is a β-defensin, e.g., a mature β-defensin. In still further embodiments, the defensin peptide is a θ-defensin, e.g., a mature θ-defensin. Preferably, the defensin peptide is a human defensin peptide, such as a human α-defensin, a human β-defensin, or a human θ-defensin, (e.g., a mature form of any of the foregoing).

Exemplary α-defensins that can be administered in accordance with the methods of the invention include human neutrophil peptide 1 (HNP1) (SEQ ID NO: 1; MRTLAILAAILLVALQAQAEPLQARADEVAAAPEQIAADIPEVVVSLAWDESLAPKH PGSRKNMACYCRIPACIAGERRYGTCIYQGRLWAFCC), human neutrophil peptide 2 (HNP2) (SEQ ID NO: 2; CYCRIPACIAGERRYGTCIYQGRLWAFCC), human neutrophil peptide 3 (HNP3) (SEQ ID NO: 3; MRTLAILAAILLVALQAQAEPLQARADEVAAAPEQIAADIPEVVVSLAWDESLAPKH PGSRKNMDCYCRIPACIAGERRYGTCIYQGRLWAFCC), human neutrophil peptide 4 (HNP4) (SEQ ID NO: 4; MRIIALLAAILLVALQVRAGPLQARGDEAPGQEQRGPEDQDISISFAWDK SSALQVSGSTRGMVCSCRLVFCRRTELRVGNCLIGGVSFTYCCTRVD), human defensin 5 (HD5) (SEQ ID NO: 5; MRTIAILAAILLVALQAQAESLQERADEATTQKQSGEDNQDLAISFAGNGL SALRTSG SQARATCYCRTGRCATRESLSGVCEISGRLYRLCCR), human defensin 6 (HD6) (SEQ ID NO: 6; MRTLTILTAVLLVALQAKAEPLQAEDDPLQAKAYEADAQEQRGANDQDFAVSFAED ASSSLRALGSTRAFTCHCRRSCYSTEYSYGTCTVMGINHRFCCL), and mature form or functional variant of the foregoing. Although administration of human α-defensin peptides is a preferred embodiment, non-human α-defensins may also be used in accordance with the methods of the invention as described herein.

Exemplary β-defensins include human β-defensin 1 (HBD-1) (SEQ ID NO: 7; MRTSYLLLFTLCLLLSEMASGGNFLTGLGHRSDHYNCVSSGGQCLYSACPIFTKIQGT CYRGKAKCCK), human β-defensin 2 (HBD-2) SEQ ID NO: 8; MRVLYLLFSFLFIFLMPLPGVFGGIGDPVTCLKSGAICHPVFCPRRYKQIGTCGLPGTK CCKKP), human β-defensin 3 (HBD-3) (SEQ ID NO: 9; MRIHYLLFALLFLFLVPVPGHGGIINTLQKYYCRVRGGRCAVLSCLPKEEQIGKCSTR GRKCCRRKK), human β-defensin 4 (HBD-4) (SEQ ID NO: 10; MQRLVLLLAISLLLYQDLPVRSEFELDRICGYGTARCRKKCRSQEYRIGRCPNTYACC LRKWDESLLNRTKP) and mature form or functional variant of the foregoing. Although administration of human β-defensin peptides is a preferred embodiment, non-human β-defensins may also be used in accordance with the methods of the invention as described herein.

Exemplary θ-defensins include human θ-defensin 1 (retrocyclin-1) (SEQ ID NO: 11; GICRCICGRGICRCICGR) human θ-defensin 2 (retrocyclin-2) (SEQ ID NO: 12; XRICRCICGRGICRCICG) and mature form or functional variant of the foregoing. Although administration of human θ-defensin peptides is a preferred embodiment, non-human θ-defensins may also be used in accordance with the methods of the invention as described herein.

The invention may use any desired defensin amino acid sequence, such as the amino acid sequence of SEQ ID NOs: 1, 2, 3, 4, 5, 6, 7, 8, 9 or 10 or a sequence having identity to SEQ ID NO: 1, 2, 3, 4, 5, 6, 7, 8, 9, 10, 11 or 12. Typically it will have at least 75% identity, at least 80%, at least 85%, at least 90%, at least 95%, at least 97%, at least 98%, or at least 99%, identity to SEQ ID NO: 1, 2, 3, 4, 5, 6, 7, 8, 9, 10, 11 or 12. Amino acid sequence identity is preferably determined using a suitable sequence alignment algorithm and default parameters, such as BLAST P (Karlin and Altschul, Proc. Natl. Acad. Sci. USA 87(6):2264-2268 (1990)).

The invention may use any desired mature defensin amino acid sequence, such as amino acids 65-94 of SEQ ID NO: 1, SEQ ID NO: 2, amino acids 65-94 of SEQ ID NO: 3, amino acids 64-97 of SEQ ID NO: 4, amino acids 63-94 of SEQ ID NO: 5, amino acids 69-100 of SEQ ID NO: 6, amino acids 33-68 of SEQ ID NO: 7, amino acids 27-64 of SEQ ID NO: 8, amino acids 22-67 of SEQ ID NO: 9, amino acids 25-72 of SEQ ID NO: 10, amino acids 1-18 of SEQ ID NO: 11, or amino acids 1-18 of SEQ ID NO: 12. In some embodiments, the defensin amino acid sequence will have at least 75%, at least 80%, at least 85%, at least 90%, at least 95%, at least 97%, at least 98%, or at least 99% identity to amino acids 65-94 of SEQ ID NO: 1, SEQ ID NO: 2, amino acids 65-94 of SEQ ID NO: 3, amino acids 64-97 of SEQ ID NO: 4, amino acids 63-94 of SEQ ID NO: 5, amino acids 69-100 of SEQ ID NO: 6, the amino acids 33-68 of SEQ ID NO: 7, amino acids 27-64 of SEQ ID NO: 8, amino acids 22-67 of SEQ ID NO: 9, amino acids 25-72 of SEQ ID NO: 10, amino acids 1-18 of SEQ ID NO: 11, or amino acids 1-18 of SEQ ID NO: 12. Amino acid sequence identity is preferably determined using a suitable sequence alignment algorithm and default parameters, such as BLAST P (Karlin and Altschul, Proc. Natl. Acad. Sci. USA 87(6):2264-2268 (1990)).

Non-enveloped, or “naked”, viruses are viruses that form without a membrane envelope surrounding the protein capsid. Exemplary non-enveloped viral pathogens that may cause a non-enveloped viral infection that may be treated in accordance with the methods of the invention include viruses of the families: Picornaviridae (e.g., coxsackievirus and rhinovirus), Adenoviridae (e.g., adenovirus), Papillomaviridae (e.g., papillomavirus), Parvoviridae (e.g., parvovirus), Reoviridae (e.g., reovirus and rotavirus), Polyomaviridae (e.g., JC virus and BK virus), Caliciviridae (e.g., calicivirus), etc. In particular embodiments, the individual is infected with a non-enveloped virus of the Picornaviridae family. In a further particular embodiment, the individual is infected with a rhinovirus. In a more particular embodiment, the individual is infected with a human rhinovirus.

In particular aspects, the invention is a method for treating or preventing rhinovirus infection and/or disease caused by rhinovirus infection, comprising administering an effective amount of a defensin peptide (i.e., one or more) to an individual in need thereof. The defensin peptide can be an α-defensin, a β-defensin, a θ-defensin or combinations thereof. In some embodiments, the defensin peptide is an α-defensin. In other embodiments, the defensin peptide is a β-defensin. In yet other embodiments, the defensin peptide is a θ-defensin. In preferred embodiments, the defensin peptide is a human defensin peptide (α-, β-, or θ-defensin). In still further preferred embodiments, the defensin peptide is a human defensin peptide selected from the group consisting of human neutrophil peptide 1 (HNP1) (SEQ ID NO: 1), human neutrophil peptide 2 (HNP2) (SEQ ID NO: 2), human neutrophil peptide 3 (HNP3) (SEQ ID NO: 3), human neutrophil peptide 4 (HNP4) (SEQ ID NO: 4), human defensin 5 (HD5) (SEQ ID NO: 5), human defensin 6 (HD6) (SEQ ID NO: 6), human β-defensin 1 (HBD-1) (SEQ ID NO: 7), human β-defensin 2 (HBD-2) (SEQ ID NO: 8), human β-defensin 3 (HBD-3) (SEQ ID NO: 9), human β-defensin 4 (HBD-4) (SEQ ID NO: 10), human θ-defensin 1 (retrocyclin-1) (SEQ ID NO: 11), human θ-defensin 2 (retrocyclin-2) (SEQ ID NO: 12) and combinations thereof.

In one aspect, the invention is a method of treating or preventing a rhinovirus infection and/or disease caused by rhinovirus infection, comprising administering an effective amount of a human neutrophil peptide 1 (HNP1) (SEQ ID NO: 1), amino acids 65-94 of SEQ ID NO:1, or a peptide having at least 85% identity to amino acids 65-94 of SEQ ID NO:1, to an individual in need thereof.

In another aspect, the invention is a method of treating or preventing a rhinovirus infection and/or disease caused by rhinovirus infection, comprising administering an effective amount of a human neutrophil peptide 2 (HNP2) (SEQ ID NO: 2), or a peptide having at least 85% identity to SEQ ID NO:2, to an individual in need thereof.

In another aspect, the invention is a method of treating or preventing a rhinovirus infection and/or disease caused by rhinovirus infection, comprising administering an effective amount of a human neutrophil peptide 3 (HNP3) (SEQ ID NO: 3), amino acids 65-94 of SEQ ID NO:3, or a peptide having at least 85% identity to amino acids 65-94 of SEQ ID NO:3, to an individual in need thereof.

In another aspect, the invention is a method of treating or preventing a rhinovirus infection and/or disease caused by rhinovirus infection, comprising administering an effective amount of a human neutrophil peptide 4 (HNP4) (SEQ ID NO: 4), amino acids 64-97 of SEQ ID NO:4, or a peptide having at least 85% identity to amino acids 64-97 of SEQ ID NO:4, to an individual in need thereof.

In another aspect, the invention is a method of treating or preventing a rhinovirus infection and/or disease caused by rhinovirus infection, comprising administering an effective amount of a formulation comprising human defensin 5 (HD5) (SEQ ID NO: 5), amino acids 63-94 of SEQ ID NO:5, or a peptide having at least 85% identity to amino acids 63-94 of SEQ ID NO:5, to an individual in need thereof.

In another aspect, the invention is a method of treating or preventing a rhinovirus infection and/or disease caused by rhinovirus infection, comprising administering an effective amount of a human defensin 6 (HD6) (SEQ ID NO: 6), amino acids 69-100 of SEQ ID NO:6, or a peptide having at least 85% sequence identity to amino acids 69-100 of SEQ ID NO:6, to an individual in need thereof.

In another aspect, the invention is a method of treating or preventing a rhinovirus infection and/or disease caused by rhinovirus infection, comprising administering an effective amount of a human β-defensin 1 (HBD-1) (SEQ ID NO: 7), amino acids 33-68 of SEQ ID NO:7, or a peptide having at least 85% sequence identity to amino acids 33-68 of SEQ ID NO:7, to an individual in need thereof.

In another aspect, the invention is a method of treating or preventing a rhinovirus infection and/or disease caused by rhinovirus infection, comprising administering an effective amount of a human β-defensin 2 (HBD-2) (SEQ ID NO: 8), amino acids 27-64 of SEQ ID NO:8, or a peptide having at least 85% sequence identity to amino acids 27-64 of SEQ ID NO:8, to an individual in need thereof.

In another aspect, the invention is a method of treating or preventing a rhinovirus infection and/or disease caused by rhinovirus infection, comprising administering an effective amount of a human β-defensin 3 (HBD-3) (SEQ ID NO: 9), amino acids 22-67 of SEQ ID NO:9, or a peptide having at least 85% sequence identity to amino acids 22-67 of SEQ ID NO:9, to an individual in need thereof.

In another aspect, the invention is a method of treating or preventing a rhinovirus infection and/or disease caused by rhinovirus infection, comprising administering an effective amount of a human β-defensin 4 (HBD-4) (SEQ ID NO: 10), amino acids 25-72 of SEQ ID NO:10, or a peptide having at least 85% sequence identity to amino acids 25-72 of SEQ ID NO:10, to an individual in need thereof.

In another aspect, the invention is a method of treating or preventing a rhinovirus infection and/or disease caused by rhinovirus infection, comprising administering an effective amount of a human θ-defensin 1 (retrocyclin-1) (SEQ ID NO: 11), or a peptide having at least 85% sequence identity to SEQ ID NO:11, to an individual in need thereof.

In another aspect, the invention is a method of treating or preventing a rhinovirus infection and/or disease caused by rhinovirus infection, comprising administering an effective amount of a human θ-defensin 2 (retrocyclin-2) (SEQ ID NO: 12), or a peptide having at least 85% sequence identity to SEQ ID NO:12, to an individual in need thereof.

In the methods described herein, preferably, the effective amount of a defensin peptide is administered to the respiratory tract of the individual (e.g., a patient with a non-enveloped viral infection).

In preferred embodiments of the methods described herein, the individual has or is at risk for acquiring a non-enveloped viral infection, such as a rhinovirus infection.

In a particularly preferred embodiment of the methods described herein, the individual has or is at risk for rhinovirus infection.

The methods of the invention can further comprise administering a co-therapeutic agent, such as mucoactive or mucolytic agents, surfactants, cough suppressants, expectorants, steroids such as a corticosteroid, bronchodilators, antihistamines, anti-inflammatory agents, antibiotics, and antivirals (e.g. pleconaril (3-{3,5-dimethyl-4-[3-(3-methylisoxazol-5-yl)propoxy] phenyl}-5-(trifluoromethyl)-1,2,4-oxadiazole) and pirodavir (ethyl 4-[2-[1-(6-methyl-3-pyridazinyl)-4-piperidinyl]ethoxy]benzoate). Co-therapeutic agents can be administered in any desired way, provided that there is overlap in the pharmacological activity of the defensin peptide and the co-therapeutic agent. For example, the co-therapeutic agent can be administered before, after or substantially concurrently with the defensin peptide.

### Formulations

Generally, the defensin peptide is administered to an individual as a component of a pharmaceutical composition. The invention relates to pharmaceutical and/or physiologically acceptable compositions which contain one or more defensin peptides as described herein. The pharmaceutical compositions comprise a defensin peptide (e.g., one or more defensin peptides) as an active ingredient and can further comprise one or more of a suitable carrier, diluent, excipient, adjuvant, and/or preservative. In some embodiments, the pharmaceutical compositions consist essentially of a defensin peptide (e.g., lyophilized or spray dried defensin polypeptide). Formulation of the defensin peptide will vary according to the route of administration selected (e.g., solution, spray, aerosol). Standard pharmaceutical formulation techniques can be employed. (See, generally, “Remington's Pharmaceutical Science,” 18^{th} Edition, Mack Publishing (1990; Baker, et al., “Controlled Release of Biological Active Agents,” John Wiley and Sons (1986), the entire teachings of both of the foregoing are incorporated by reference.)

The pharmaceutical composition can contain antibacterial and/or antifungal agents, for example, parabens, chlorobutanol, alcohols (e.g., phenol, benzyl alcohol), sorbic acid, and the like. The pharmaceutical composition can contain suitable agents for adjusting tonicity, for example sugars, sodium chloride, and the like.

The pharmaceutical composition can be in a dosage form appropriate to the desired route of administration.

Dosage forms for topical and local administration include powders, sprays, drops, gels, foams, liquids and inhalants, and these may be formulated to dispense as a single dose or multiple doses. The defensin peptide can be admixed under suitable conditions (e.g., sterile conditions) with a physiologically acceptable carrier and any preservatives, buffers, or propellants as may be required.

For inhalation, the pharmaceutical composition can be, for example, a liquid or a dry powder and can be loaded into a suitable dispenser or device for administration (e.g., an atomizer, nebulizer, pressurized aerosol dispenser, dry powder inhaler, metered dose inhaler and the like). For intranasal administration, the pharmaceutical composition can be, for example, a liquid, a dry powder, a gel or foam, and can be loaded into a suitable dispenser or device for administration, for example as aerosols, drops or insufflations. Compositions for administration as nasal drops may contain one or more excipients of the type usually included in such compositions, for example preservatives, viscosity adjusting agents, tonicity adjusting agents, pH buffering agents and the like.

Solid dosage forms include, for example, capsules, tablets, pills, powders, and granules. In such solid dosage forms, the active ingredient (i.e., one or more defensin peptides) can be admixed with one or more (a) carrier or excipient such as sodium citrate or dicalcium phosphate; (b) fillers or extenders, for example, starches, lactose, sucrose, glucose, mannitol, silicic acid, polyethyleneglycols, and the like; (c) binders, for example, carboxymethylcellulose, alginates, gelatin, polyvinylpyrrolidone, sucrose, and acacia; (d) humectants, for example, glycerol; (e) disintegrating agents, for example, agar-agar, calcium carbonate, potato or tapioca starch, alginic acid, certain complex silicates, and sodium carbonate; (f) solution retarders, for example paraffin; (g) absorption accelerators, for example, quaternary ammonium compounds; (h) wetting agents, for example, cetyl alcohol, and glycerol monostearate; (i) adsorbents, for example, kaolin and bentonite; and (j) lubricants, for example, talc, calcium stearate, magnesium stearate, solid polyethylene glycols, sodium lauryl sulfate, or mixtures thereof. Solid compositions, such as those for oral administration, can also comprise buffering agents. Such solid compositions or solid compositions that are similar to those described can be provided in soft- or hard-filled gelatin capsules if desired.

Solid dosage forms such as tablets, dragées, capsules, pills, and granules can be prepared with coatings and shells, such as enteric coatings or other suitable coatings or shells. Several such coatings and/or shells are well known in the art, and can contain opacifying agents, and can also be of such composition that they release the active compound or compounds in a certain part of the intestinal tract in a delayed manner. Examples of embedding compositions which can be used are polymeric substances and waxes. The active compounds can also be used in microencapsulated form, if appropriate, with, for example, one or more of the above-mentioned carriers or excipients.

Liquid dosage forms include pharmaceutically acceptable emulsions, solutions, suspensions, syrups, and elixirs. In addition to the active defensin peptide, the liquid dosage forms can contain a suitable carrier or excipient, such as water or other solvents, solubilizing agents and emulsifiers as, for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like. If desired, the composition can also include wetting agents, emulsifying agents, suspending agents, sweetening, flavoring and/or perfuming agents. Suspensions can contain suspending agents, such as, ethoxylated isostearyl alcohols, polyoxyethylene sorbitol and sorbitan esters, microcrystalline cellulose, aluminum metahydroxide, bentonite, agar-agar, tragacanth, and the like. Mixtures of suspending agents can be employed if desired.

Compositions suitable for parenteral administration can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, excipients or vehicles include physiological saline, phosphate-buffered saline, Hank's solution, Ringer's-lactate and the like, ethanol, polyols (propyleneglycol, polyethyleneglycol, glycerol, and the like), vegetable oils (such as olive oil) and injectable organic esters such as ethyl oleate, or any suitable mixture thereof. Fluidity can be adjusted, for example, by the use of a coating such as lecithin, by the maintenance of the required particle size in the case of dispersions and by the use of surfactants. When prolonged absorption of an injectable pharmaceutical composition is desired, agents that delay absorption, for example, aluminum monostearate and gelatin can be included.

The quantity of active ingredient (one or more defensin peptides) in the composition can range from about 0.1% to about 99.9% by weight. Preferably the quantity of active ingredient is about 10% to about 90%, or about 20% to about 80% by weight. A unit dose preparation can contain from about 1 mg to about 1000 mg active ingredient, preferably about 10 mg to about 100 mg active ingredient. The composition can, if desired, also contain other compatible therapeutic agents, such as mucoactive or mucolytic agents, surfactants, cough suppressants, expectorants, steroids such as a corticosteroid, bronchodilators, antihistamines, anti-inflammatory agents, antibiotics, antivirals, and the like. Antivirals include, for example, pleconaril (3-{3 ,5-dimethyl-4-[3-(3-methylisoxazol-5-yl)propoxy] phenyl}-5-(trifluoromethyl)-1,2,4-oxadiazole, Schering-Plough Corp., Kenilworth, N.J.) (Florea N, Maglio D, Nicolau D (2003). Pleconaril, a novel antipicornaviral agent. Pharmacotherapy 23 (3): 339-48) and pirodavir (R77975, ethyl 4-[2-[1-(6-methyl-3-pyridazinyl)-4-piperidinyl]ethoxy]benzoate) (Hayden F G, Andries K, Janssen P A (1992). Safety and efficacy of intranasal pirodavir (R77975) in experimental rhinovirus infection. Antimicrob. Agents Chemother. 36(4): 727-732).

The defensin peptide formulation may comprise one or more defensin peptides. In some embodiments, one or more different defensin peptides are co-administered. In some embodiments, the defensin peptide formulation comprises one or more α-defensin peptides and one or more β-defensin peptides. In other embodiments, the defensin peptide formulation comprises one or more α-defensin peptides and one or more θ-defensin peptides. In yet other embodiments, the defensin peptide formulation comprises one or more β-defensin peptides and one or more θ-defensin peptides. In another embodiment, the defensin peptide formulation comprises one or more α-defensin peptides, one or more β-defensin peptides, and one or more θ-defensin peptides.

In one embodiment, the composition comprises one or more defensin peptides (α-, β-, or θ-defensin). In another embodiment, the composition comprises one or more defensin peptides (α-, β-, or θ-defensin) and a physiologically acceptable carrier or excipient.

In one embodiment, the composition comprises one or more α- or β-defensin peptides. In another embodiment, the composition comprises one or more α- or β-defensin peptides and a physiologically acceptable carrier or excipient.

In one embodiment, the composition comprises one or more α-defensin peptides. In another embodiment, the composition comprises one or more α-defensin peptide and a physiologically acceptable carrier or excipient.

In one embodiment, the composition comprises one or more β-defensin peptides. In another embodiment, the composition comprises one or more β-defensin peptides and a physiologically acceptable carrier or excipient.

In one embodiment, the composition comprises one or more θ-defensin peptides. In another embodiment, the composition comprises one or more θ-defensin peptides and a physiologically acceptable carrier or excipient.

In one embodiment, the composition comprises a fusion protein of one or more defensin peptides. In one embodiment, the fusion protein comprises one or more domain of one defensin and one or more domain of a different defensin. For example, synthetic defensin fusion proteins can be generated that comprise domains (e.g. internal domains and/or N-/C-terminal domains) from hBD1 and hBD3, which exhibit antimicrobial activity and high salt tolerance. (Scudiero O et al. (2010). Novel Synthetic, Salt-Resistant Analogs of Human Beta-Defensins 1 and 3 Endowed with Enhanced Antimicrobial Activity. Antimicrob Agents Chemother. 54(6): 2312-2322).

In another embodiment, the composition comprises a fusion protein comprising a solubility-enhancing moiety useful, e.g. for enhanced recovery of defensins in recombinant expression systems. For example, an N-terminal fragment of a silk spider protein can be used to confer enhanced solubility, as described in PCT Publication No. WO 2011/115538.

In one embodiment, the composition comprises human neutrophil peptide 1 (HNP1) (SEQ ID NO: 1), amino acids 65-94 of SEQ ID NO:1, or a peptide having at least 85% identity to amino acids 65-94 of SEQ ID NO:1. In another embodiment, the composition comprises human neutrophil peptide 1 (HNP1) (SEQ ID NO: 1), amino acids 65-94 of SEQ ID NO:1, or a peptide having at least 85% identity to amino acids 65-94 of SEQ ID NO:1 and a physiologically acceptable carrier or excipient.

In one embodiment, the composition comprises human neutrophil peptide 2 (HNP2) (SEQ ID NO: 2), or a peptide having at least 85% identity to SEQ ID NO:2. In another embodiment, the composition comprises human neutrophil peptide 2 (HNP2) (SEQ ID NO: 2), or a peptide having at least 85% identity to SEQ ID NO:2, and a physiologically acceptable carrier or excipient.

In one embodiment, the composition comprises human neutrophil peptide 3 (HNP3) (SEQ ID NO: 3), amino acids 65-94 of SEQ ID NO:3, or a peptide having at least 85% identity to amino acids 65-94 of SEQ ID NO:3. In another embodiment, the composition comprises human neutrophil peptide 3 (HNP3) (SEQ ID NO: 3), amino acids 65-94 of SEQ ID NO:3, or a peptide having at least 85% identity to amino acids 65-94 of SEQ ID NO:3, and a physiologically acceptable carrier or excipient.

In one embodiment, the composition comprises human neutrophil peptide 4 (HNP4) (SEQ ID NO: 4), amino acids 64-97 of SEQ ID NO:4, or a peptide having at least 85% identity to amino acids 64-97 of SEQ ID NO:4. In another embodiment, the composition comprises human neutrophil peptide 4 (HNP4) (SEQ ID NO: 4), amino acids 64-97 of SEQ ID NO:4, or a peptide having at least 85% identity to amino acids 64-97 of SEQ ID NO:4, and a physiologically acceptable carrier or excipient.

In one embodiment, the composition comprises human defensin 5 (HD5) (SEQ ID NO: 5), amino acids 63-94 of SEQ ID NO:5, or a peptide having at least 85% identity to amino acids 63-94 of SEQ ID NO:5. In another embodiment, the composition comprises human defensin 5 (HD5) (SEQ ID NO: 5), amino acids 63-94 of SEQ ID NO:5, or a peptide having at least 85% identity to amino acids 63-94 of SEQ ID NO:5, and a physiologically acceptable carrier or excipient.

In one embodiment, the composition comprises human defensin 6 (HD6) (SEQ ID NO: 6), amino acids 69-100 of SEQ ID NO:6, or a peptide having at least 85% sequence identity to amino acids 69-100 of SEQ ID NO:6. In another embodiment, the composition comprises human defensin 6 (HD6) (SEQ ID NO: 6), amino acids 69-100 of SEQ ID NO:6, or a peptide having at least 85% sequence identity to amino acids 69-100 of SEQ ID NO:6, and a physiologically acceptable carrier or excipient.

In one embodiment, the composition comprises human β-defensin 1 (HBD-1) (SEQ ID NO: 7), amino acids 33-68 of SEQ ID NO:7, or a peptide having at least 85% sequence identity to amino acids 33-68 of SEQ ID NO:7. In another embodiment, the composition comprises human β-defensin 1 (HBD-1) (SEQ ID NO: 7), amino acids 33-68 of SEQ ID NO:7, or a peptide having at least 85% sequence identity to amino acids 33-68 of SEQ ID NO:7, and a physiologically acceptable carrier or excipient.

In one embodiment, the composition comprises human β-defensin 2 (HBD-2) (SEQ ID NO: 8), amino acids 27-64 of SEQ ID NO:8, or a peptide having at least 85% sequence identity to amino acids 27-64 of SEQ ID NO:8. In another embodiment, the composition comprises human β-defensin 2 (HBD-2) (SEQ ID NO: 8), amino acids 27-64 of SEQ ID NO:8, or a peptide having at least 85% sequence identity to amino acids 27-64 of SEQ ID NO:8, and a physiologically acceptable carrier or excipient.

In one embodiment, the composition comprises human β-defensin 3 (HBD-3) (SEQ ID NO: 9), amino acids 22-67 of SEQ ID NO:9, or a peptide having at least 85% sequence identity to amino acids 22-67 of SEQ ID NO:9. In another embodiment, the composition comprises human β-defensin 3 (HBD-3) (SEQ ID NO: 9), amino acids 22-67 of SEQ ID NO:9, or a peptide having at least 85% sequence identity to amino acids 22-67 of SEQ ID NO:9, and a physiologically acceptable carrier or excipient.

In one embodiment, the composition comprises human β-defensin 4 (HBD-4) (SEQ ID NO: 10), amino acids 25-72 of SEQ ID NO:10, or a peptide having at least 85% sequence identity to amino acids 25-72 of SEQ ID NO:10. In another embodiment, the composition comprises human β-defensin 4 (HBD-4) (SEQ ID NO: 10), amino acids 25-72 of SEQ ID NO:10, or a peptide having at least 85% sequence identity to amino acids 25-72 of SEQ ID NO:10, and a physiologically acceptable carrier or excipient.

In one embodiment, the composition comprises human θ-defensin 1 (retrocyclin-1) (SEQ ID NO: 11), or a peptide having at least 85% sequence identity to SEQ ID NO:11. In another embodiment, the composition comprises human θ-defensin 1 (retrocyclin-1) (SEQ ID NO: 11), amino acids 1-18 of SEQ ID NO:11, or a peptide having at least 85% sequence identity to SEQ ID NO:11, and a physiologically acceptable carrier or excipient.

In one embodiment, the composition comprises human θ-defensin 2 (retrocyclin-2) (SEQ ID NO: 12), or a peptide having at least 85% sequence identity to SEQ ID NO:12. In another embodiment, the composition comprises human θ-defensin 2 (retrocyclin-2) (SEQ ID NO: 12), or a peptide having at least 85% sequence identity to SEQ ID NO:12, and a physiologically acceptable carrier or excipient.

### Modes of Administration

The defensin peptide or formulation can be administered in any suitable way. Preferably, the selected mode of administration results in the defensin peptide accumulating to an effective amount in tissue(s) where non-enveloped viruses enter host cells and/or replicate. Suitable modes of administration include, for example, parenterally (e.g., intravenous, intramuscular, intraperotineal, intratracheal, or subcutaneous injection), topically, locally (e.g., intranasally or by inhalation), or orally. The defensin peptide can be administered in a single dose or multiple doses as indicated.

In some embodiments, the defensin peptide is administered locally to a mucosal surface of the body, such as the respiratory tract, gastrointestinal tract (e.g., mouth, esophagus, stomach, intestines, bowel, anus), skin (e.g., epidermis), or urogenital tract (e.g., uterine, vaginal, endometrial, penile). Preferably, the defensin peptide is administered topically or locally to the respiratory tract (e.g., to the mucosal surface of the respiratory tract, including the nasal passages, sinuses, trachea, lungs, etc.), and can be administered in any suitable form, such as a solution, a suspension, a spray, a mist, a foam, a gel, a vapor, droplets, particles, or a dry powder. In more preferred embodiments, the defensin peptide is aerosolized (e.g., an aerosolized liquid or dry powder) for administration to the respiratory tract, including the nasal passages, sinuses, trachea, and/or lungs. The defensin peptide can be aerosolized for administration via the oral airways using any suitable method and/or device, and many suitable methods and devices are conventional and well-known in the art. For example, the defensin peptide can be aerosolized using a suitable inhaler such as a dry powder inhaler (DPI) or metered dose inhaler (MDI) (e.g., a pressurized metered dose inhaler (pMDI) including HFA propellant, or a non-HFA propellant) with or without a spacer or holding chamber, a nebulizer, an atomizer, a continuous sprayer, an oral spray. The defensin peptide can be aerosolized for administration via the nasal airways using, for example, a nasal pump or sprayer, a DPI, MDI, pMDI with or without a spacer or holding chamber, a nebulizer with or without a nasal adapter or prongs, an atomizer, or a continuous sprayer. The defensin peptide can also be delivered to the nasal mucosal surface via, for example, nasal wash, intranasal spray, or gel, and to the oral mucosal surfaces via, for example, an oral wash, spray, or gel. The defensin peptide can be delivered to the mucosal surfaces of the sinuses via, for example, intranasal sprays, nebulizers with nasal adapters and nasal nebulizers with oscillating or pulsatile airflows.

The term “aerosol”, as used herein, refers to any preparation of a fine mist of particles (including liquid and non-liquid particles, e.g., dry powders), typically with a volume median geometric diameter of about 0.1 to about 30 microns or a mass median aerodynamic diameter of between about 0.5 and about 10 microns. Preferably the volume median geometric diameter for the aerosol particles is less than about 10 microns. The preferred volume median geometric diameter for aerosol particles is about 5 microns. For example, the aerosol can contain particles that have a volume median geometric diameter between about 0.1 and about 30 microns, between about 0.5 and about 20 microns, between about 0.5 and about 10 microns, between about 1.0 and about 3.0 microns, between about 1.0 and 5.0 microns, between about 1.0 and 10.0 microns, between about 5.0 and 15.0 microns. Preferably the mass median aerodynamic diameter is between about 0.5 and about 10 microns, between about 1.0 and about 3.0 microns, or between about 1.0 and 5.0 microns.

The geometry of the airways is an important consideration when selecting a suitable method for producing and delivering aerosols to the lungs. The lungs are designed to entrap particles of foreign matter that are breathed in, such as dust. There are three basic mechanisms of deposition: impaction, sedimentation, and Brownian motion (J. M. Padfield. 1987. In: D. Ganderton & T. Jones eds. Drug Delivery to the Respiratory Tract, Ellis Harwood, Chicherster, U.K.). Impaction in the upper airways occurs when particles are unable to stay within the air stream, particularly at airway branches. Impacted particles are adsorbed onto the mucus layer covering bronchial walls and eventually cleared from the lungs by mucociliary action. Impaction mostly occurs with particles over 5 μm in aerodynamic diameter. Smaller particles (those less than about 3 μm in aerodynamic diameter) tend to stay within the air stream and to be advected deep into the lungs. Sedimentation often occurs in the lower respiratory system where airflow is slower. Very small particles (those less than about 0.6 μm) can deposit by Brownian motion. Deposition by Brownian motion is generally undesirable because deposition cannot be targeted to the alveoli (N. Worakul & J. R. Robinson. 2002. In: Polymeric Biomaterials, 2^{nd} Ed. S. Dumitriu ed. Marcel Dekker. New York).

For administration to the respiratory tract by inhalation, a suitable method (e.g., nebulization, dry powder inhaler) is selected to produce aerosols with the appropriate particle size for preferential delivery to the desired region of the respiratory tract, such as the deep lung (generally particles between about 0.6 microns and 5 microns in diameter), the upper airway (generally particles of about 3 microns or larger diameter), the deep lung and the upper airway, or the nasal mucosal surfaces.

In the methods of the invention, an “effective amount” of a defensin peptide is administered to an individual in need thereof. An effective amount is an amount that is sufficient to achieve the desired therapeutic or prophylactic effect, such as an amount sufficient to reduce non-enveloped viral infectivity or infection (e.g., a rhinovirus infection), to reduce duration of illness, to reduce pathogen burden, to reduce the number of days that infected individuals experience viral infection symptoms and/or to decrease the incidence or rate of viral infection.

The clinician of ordinary skill can determine appropriate dosage based on the properties of the particular defensin peptide selected and other conventional factors, for example, the individual's age, sensitivity or tolerance to drugs, the particular infection to be treated and the individual's overall well-being, and the treating clinician's sound judgment.

### EXEMPLIFICATION

### Example 1

### β-Defensins Reduce the Infectivity of Rhinovirus in a Dose-Dependent Manner.

To determine if β-defensins had antiviral activity against rhinovirus, recombinant human β-defensins (Peprotech, Rocky Hill, N.J.) were used in assays with rhinovirus (Rv16). Recombinant β-defensin proteins were diluted in PBS to the desired concentrations (0.25-1.25 μg/mL). Virus was added to each tube to a final volume of 40 μL and incubated for 1 hour at 37° C. plus 5% CO₂. After incubation, 200 uL of cell culture media was added to each tube and viral titers were quantified in a 50% Tissue Culture Infectious Dose (TCID₅₀) assay using H1-HeLa cells. The TCID₅₀ assay is a standard endpoint dilution assay that is used to quantify how much of a given virus is present in a sample. The limit of detection for rhinovirus in the assay was 1.2 log₁₀ TCID₅₀.

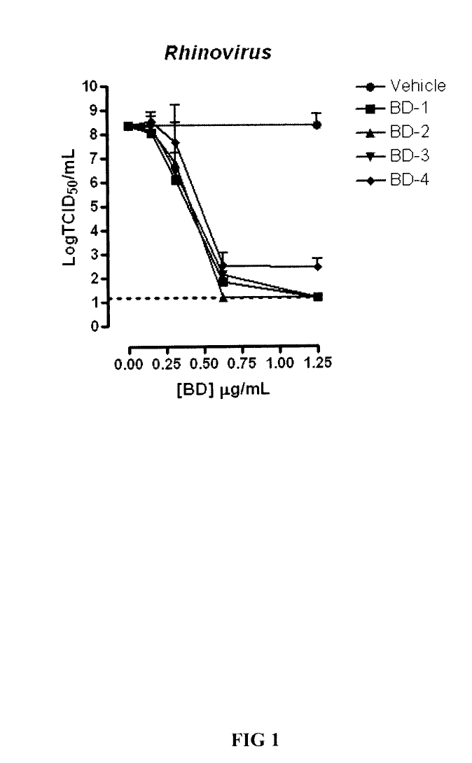

Surprisingly, all four of the β-defensins tested reduced the infectivity of rhinovirus in a dose-responsive manner in two independent experiments (FIG. 1). Each of the β-defensins exhibited comparable potency and significantly reduced viral infectivity at concentrations greater than 0.3125 μg/mL, with a maximal reduction in viral titer of greater than 10⁷. These results indicated that, unexpectedly, β-defensins (i.e., HBD-1, HBD-2, HBD-3 and HBD-4) significantly reduced viral concentration and thus, had a direct anti-viral effect.

### Example 2

### α-Defensins Exhibit Dose-Dependent Antiviral Activity Against Rhinovirus

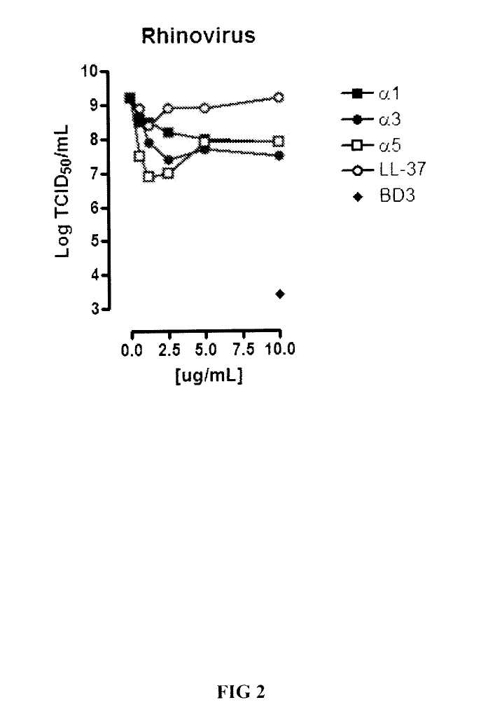

Human α-defensins and LL-37, a broadly expressed antimicrobial peptide from the cathelicidin family, were also tested for antiviral activity against rhinovirus. Recombinant human α-defensins (HNP-1, HNP-3 and HNP-5) (Pepnet, Louisville, Ky.) and recombinant LL-37 (Pepnet, Louisville, Ky.) were used in assays with rhinovirus (Rv16) to determine their antiviral activity. Recombinant HBD-3 (Peprotech, Rocky Hill, N.J.) was used as a control. The recombinant proteins were diluted in PBS to the desired concentrations (see FIG. 2). Virus was added to each tube to a final volume of 40 μL and incubated for 1 hour at 37° C. plus 5% CO₂. After incubation, 200 μL of cell culture media was added to each tube and the concentration of virus was quantified by TCID₅₀ assay using H1-HeLa cells. The limit of detection for rhinovirus in the assay was 1.2 log₁₀ TCID₅₀/mL.

Of the α-defensins, HNP-5 was the most effective and reduced rhinovirus titers by 2.5 Log₁₀ TCID₅₀/mL at 2 μg/mL, while HNP-3 and HNP-1 reached maximal efficacy at 2.5 μg/mL and 5 μg/mL (2.3 Log₁₀ and 1 Log₁₀ reduction), respectively (FIG. 2). LL-37 had little effect (maximal efficacy 0.5 Log₁₀ TCID₅₀/mL reduction at 1.25 μg/mL) compared to the α-defensins. All three of the α-defensins and LL-37 reached maximal efficacy at lower concentrations and were less effective at higher concentrations. Collectively, the data indicated that although rhinovirus was most susceptible to β-defensins (see FIG. 2, HBD-3), it was also susceptible to α-defensins.

1. A method of treating or preventing non-enveloped virus infection and/or disease caused by non-enveloped virus infection, comprising administering to an individual in need thereof an effective amount of a human defensin peptide, wherein said non-enveloped virus is human rhinovirus.

2. (canceled)

3. The method of claim 1, wherein said defensin peptide is a human α-defensin peptide, a human β-defensin peptide or combinations thereof.

4. (canceled)

5. The method of claim 1, wherein said human defensin peptide is selected from the group consisting of human β-defensin 2 (HBD-2), human neutrophil peptide 1 (HNP1) (SEQ ID NO: 1), human neutrophil peptide 2 (HNP2) (SEQ ID NO: 2), human neutrophil peptide 3 (HNP3) (SEQ ID NO: 3), human neutrophil peptide 4 (HNP4) (SEQ ID NO: 4), human defensin 5 (HD5) (SEQ ID NO: 5), human defensin 6 (HD6) (SEQ ID NO: 6), human β-defensin 1 (HBD-1) (SEQ ID NO: 7), human β-defensin 3 (HBD-3) (SEQ ID NO: 9), human β-defensin 4 (HBD-4) (SEQ ID NO: 10), and combinations thereof.

6. The method of claim 1, wherein said human defensin peptide is selected from the group consisting of human β-defensin 2 (HBD-2) (amino acids 27-64 of SEQ ID NO:8), human neutrophil peptide 1 (HNP1) (amino acids 65-94 of SEQ ID NO: 1), human neutrophil peptide 3 (HNP3) (amino acids 65-94 of SEQ ID NO: 3), human neutrophil peptide 4 (HNP4) (amino acids 64-97 of SEQ ID NO: 4), human defensin 5 (HD5) (amino acids 63-94 of SEQ ID NO: 5), human defensin 6 (HD6) (amino acids 69-100 of SEQ ID NO: 6), human β-defensin 1 (HBD-1) (amino acids 33-68 of SEQ ID NO: 7), human β-defensin 3 (HBD-3) (amino acids 22-67 of SEQ ID NO: 9), human β-defensin 4 (HBD-4) (amino acids 25-72 of SEQ ID NO: 10), and combinations thereof.

7. The method of claim 1, wherein said human defensin peptide is a human α-defensin peptide.

8. (canceled)

9. The method of claim 1, wherein said human α-defensin peptide is selected from the group consisting of human neutrophil peptide 1 (HNP1) (SEQ ID NO: 1), human neutrophil peptide 2 (HNP2) (SEQ ID NO: 2), human neutrophil peptide 3 (HNP3) (SEQ ID NO: 3), human neutrophil peptide 4 (HNP4) (SEQ ID NO: 4), human defensin 5 (HD5) (SEQ ID NO: 5), and human defensin 6 (HD6) (SEQ ID NO: 6).

10. The method of claim 1, wherein said human α-defensin peptide is selected from the group consisting of human neutrophil peptide 1 (HNP1) (amino acids 65-94 of SEQ ID NO: 1), human neutrophil peptide 3 (HNP3) (amino acids 65-94 of SEQ ID NO: 3), human neutrophil peptide 4 (HNP4) (amino acids 64-97of SEQ ID NO: 4), human defensin 5 (HD5) (amino acids 63-94 of SEQ ID NO: 5), and human defensin 6 (HD6) (amino acids 69-100 of SEQ ID NO: 6).

11. The method of claim 1, wherein said human defensin peptide is a human β-defensin peptide.

12. (canceled)

13. The method of claim 11, wherein said human β-defensin is selected from the group consisting of human β-defensin 1 (HBD-1) (SEQ ID NO: 7), human β-defensin 2 (HBD-2) (SEQ ID NO: 8), human β-defensin 3 (HBD-3) (SEQ ID NO: 9), and human β-defensin 4 (HBD-4) (SEQ ID NO: 10).

14. The method of claim 11, wherein said human β-defensin is selected from the group consisting of human β-defensin 1 (HBD-1) (amino acids 33-68 of SEQ ID NO: 7), human β-defensin 2 (HBD-2) (amino acids 27-64 of SEQ ID NO: 8), human β-defensin 3 (HBD-3) (amino acids 22-67 of SEQ ID NO: 9), and human β-defensin 4 (HBD-4) (amino acids 25-72 of SEQ ID NO: 10).

15. The method of claim 1 , wherein said human defensin peptide is a human θ-defensin peptide.

16. (canceled)

17. The method of claim 15, wherein said human θ-defensin peptide is selected from the group consisting of human θ-defensin 1 (retrocyclin-1) (SEQ ID NO: 11) and human θ-defensin 2 (retrocyclin-2) (SEQ ID NO: 12).

18-52. (canceled)

53. A method of treating or preventing acute exacerbation of a chronic respiratory disease, comprising administering an effective amount of a defensin peptide to an individual in need thereof.

54. The method of claim 53, wherein said chronic respiratory disease is selected from the group consisting of chronic obstructive pulmonary disease, asthma, cystic fibrosis and rhinitis.

55. The method of claim 1, wherein the defensin is administered by inhalation or intranasally.

56. The method of claim 1, wherein the defensin is administered by inhalation.

57. The method of of claim 1, wherein the defensin is administered intranasally.

58. The method of claim 53, wherein said defensin peptide is selected from the group consisting of human β-defensin 2 (HBD-2) (SEQ ID NO: 8), human neutrophil peptide 1 (HNP1) (SEQ ID NO: 1), human neutrophil peptide 2 (HNP2) (SEQ ID NO: 2), human neutrophil peptide 3 (HNP3) (SEQ ID NO: 3), human neutrophil peptide 4 (HNP4) (SEQ ID NO: 4), human defensin 5 (HD5) (SEQ ID NO: 5), human defensin 6 (HD6) (SEQ ID NO: 6), human β-defensin 1 (HBD-1) (SEQ ID NO: 7), human β-defensin 3 (HBD-3) (SEQ ID NO: 9), human β-defensin 4 (HBD-4) (SEQ ID NO: 10), and combinations thereof.

59. The method of claim 53, wherein said defensin peptide is selected from the group consisting of human β-defensin 2 (HBD-2) (amino acids 27-64 of SEQ ID NO:8), human neutrophil peptide 1 (HNP1) (amino acids 65-94 of SEQ ID NO: 1), human neutrophil peptide 3 (HNP3) (amino acids 65-94 of SEQ ID NO: 3), human neutrophil peptide 4 (HNP4) (amino acids 64-97 of SEQ ID NO: 4), human defensin 5 (HD5) (amino acids 63-94 of SEQ ID NO: 5), human defensin 6 (HD6) (amino acids 69-100 of SEQ ID NO: 6), human β-defensin 1 (HBD-1) (amino acids 33-68 of SEQ ID NO: 7), human β-defensin 3 (HBD-3) (amino acids 22-67 of SEQ ID NO: 9), human β-defensin 4 (HBD-4) (amino acids 25-72 of SEQ ID NO: 10), and combinations thereof.
